(19) United States
(12) Patent Application Publication
(10) Pub. No.: US 2014/0323534 A1
(43) Pub. Date: Oct. 30, 2014

(54) USES OF 3'-DESFERRITHIOCIN ANALOGS

(71) Applicant: University of Florida Research Foundation, Inc., Gainesville, FL (US)

(72) Inventor: Raymond J. Bergeron, JR., Gainesville, FL (US)

(73) Assignee: University of Florida Research Foundation, Inc., Gainesville, FL (US)

(21) Appl. No.: 14/363,952

(22) PCT Filed: Dec. 14, 2012
(86) PCT No.: PCT/US2012/069826

§ 371 Date: Jun. 9, 2014

Related U.S. Application Data
(60) Provisional application No. 61/576,913, filed on Dec. 16, 2011.

(51) Int. Cl. C07D 277/12

(52) U.S. Cl. CPC C07D 277/12
USPC 514/365

(57) ABSTRACT

Macular degeneration, closed head injury, stroke, irritable bowel disease, and reperfusion injury are all associated with biological injury due to reactive oxygen species, probably due to focal iron overload in many instances. The present invention provides methods and pharmaceutical compositions for treating these diseases and conditions using desferrithiocin analogs of Formula (I). In certain embodiments, the analogs include a polyether moiety at the 3′-position of the phenyl ring of the compound. Formula (I).

### RELATED APPLICATIONS

The present application claims priority under 35 U.S.C. §119(e) to U.S. provisional patent application, U.S. Ser. No. 61/576,913, filed Dec. 16, 2011, which is incorporated herein by reference.

### GOVERNMENT SUPPORT

This invention was made with U.S. Government support under grant number R37DK049108 awarded by National Institute of Diabetes and Digestive and Kidney Diseases of the National Institutes of Health. The U.S. Government has certain rights in the invention.

### BACKGROUND

Iron metabolism in primates is characterized by a highly efficient recycling process. Brittenham, “Disorders of Iron Metabolism: Iron Deficiency and Overload” In Hematology: Basic Principles and Practice; 3rd ed.; Hoffman et al., Eds.; Churchill Livingstone: New York, 2000; 397-428. Consequently, there is no specific mechanism for eliminating this transition metal. Because of the lack of an iron clearance mechanism, the introduction of “excess iron” into this closed metabolic loop often leads to chronic iron overload and can ultimately lead to biological damage (e.g., peroxidative tissue damage). There are a number of ways in which excess iron is introduced, including a high-iron diet, acute iron ingestion, or malabsorption of the metal. Conrad et al. “Iron Absorption and Transport” Am. J. Med. Sci. 1999, 318:213-229; Lieu et al. “The Roles of Iron in Health and Disease” Mol. Aspects Med. 2001, 22:1-87. In each of these situations, a subject can be treated by phlebotomy to reduce iron levels. However, for iron-overload syndromes resulting from chronic transfusion therapy, e.g., aplastic anemia and thalassemia (Olivieri et al. “Iron-chelating Therapy and the Treatment of Thalassemia” Blood 1997, 89:739-761; Vichinsky, “Current Issues with Blood Transfusions in Sickle Cell Disease” Semin. Hematol. 2001, 38:14-22; Kersten et al. “Long-Term Treatment of Transfusional Iron Overload with the Oral Iron Chelator Deferiprone (L1): A Dutch Multicenter Trial” Ann. Hematol. 1996, 73:247-252), phlebotomy is not an option. In these secondary iron overload syndromes, the origin of the excess iron is the transfused red blood cells. Since removing the red blood cells to remedy the iron overload would be counterproductive, an alternative method of removing iron is chelation therapy.

Although considerable effort has been invested in the development of new therapeutics for managing iron overload resulting from thalassemia, particularly therapeutics that can be administered orally, desferrioxamine B, a hexacoordinate hydroxamate iron chelator produced by Streptomyces pilosus, is still the drug of choice. However, desferrioxamine B is not ideal for chelation therapy because iron is removed with a low efficiency. In addition, the oral activity of desferrioxamine B is marginal, thereby requiring parenteral administration, which can result in poor patient compliance, particularly for patients in need of long-term chelation therapy.

In recent years, a substantial number of synthetic metal chelators have been studied as potential orally active therapeutic agents, e.g., pyridoxal isonicotinoyl hydrazone (PIH), hydroxypyridones and N,N′-bis-(2-hydroxybenzylethylenediamine)-N,N′-diacetic acid (HBED); however, these synthetic chelators have not yet demonstrated the desired properties for an ideal metal chelator therapeutic (e.g., effective chelation, suitable oral activity, and acceptable toxicity). Siderophores including enterobactin and rhodotorulic acid have also been studied. However, both enterobactin and rhodotorulic acid exhibit unacceptable toxicity, and neither demonstrated measurable oral activity. In general, although a large number of siderophores and synthetic iron chelators have been developed, most have been abandoned because their properties are not suitable for use in treating chronic iron overload.

The thiazoline-based siderophore desferrithiocin, isolated from Streptomyces antibioticus, has also been studied. Desferrithiocin analogs, including desazadesferrithiocin and desferrithiocin polyether analogs, have been investigated as orally active therapeutic agents for treating iron overload. The work on such analogs is described in International PCT Applications, PCT/US99/19691, filed Aug. 31, 1999; PCT/US2003/028304, filed Sep. 9, 2003; PCT/US2006/010945, filed Mar. 22, 2006; and PCT/US2008/003433, filed Mar. 14, 2008; each of which is incorporated herein by reference. These analogs have been found useful in treating diseases associated with global iron overload, such as that resulting from chronic transfusion therapy used to treat thalassemia and other transfusion-dependent anemias. Phase 2 clinical trials studying the safety and efficacy of a desferrithiocin analog in iron overload patients are ongoing.

Although not typically associated with iron overload, diseases including macular degeneration, stroke, irritable bowel disease, closed head injury, and reperfusion injury are all diseases associated with significant morbidity and mortality. For instance, macular degeneration results in the loss of central vision and is a major cause of blindness and visual impairment in older adults. Subjects with macular degeneration frequently cannot read or recognize faces due to their visual impairment. Stroke is caused by a lack of blood flow to an area of the brain and depending on the area of the brain affected can result in the inability to move limbs on one side of the body or can affect speech or vision. Reperfusion injury is due to oxidative stress in ischemic tissue after blood flow has been restored. Irritable bowel disease (IBD) is a functional bowel disease characterized by abdominal pain and discomfort, bloating, diarrhea, and/or constipation in the absence of any detectable cause. Although IBD does not lead to more serious problems in most patients, it is a source of chronic pain and fatigue for patients who suffer with this condition. And finally closed head injury is the leading cause of death in children under 4 years of age and is the most common cause of physical disability and cognitive impairment in young people. All of these diseases need better treatments including new approaches to their treatment.

### SUMMARY OF THE INVENTION

The present invention stems from the recognition that the pathogenesis of various diseases, including macular degeneration, closed head injury, irritable bowel disease (IBD), stroke, reperfusion injury, and other diseases and conditions, involves free iron and the generation of reactive oxygen species (ROS), including superoxide anion, hydrogen peroxide, hypochlorous acid, and hydroxyl radicals, and other longer lived, free radicals. Such radicals are now realized to be important contributors to many diseases including macular degeneration, head injury, IBD, stroke, and reperfusion injury. As appreciated in the art, free iron contributes to the formation of reactive oxygen species. For example, Fe⁺² ions in biological systems react with oxygen species to produce highly reactive hydroxyl radicals via the Fenton reaction (see scheme below). The hydroxyl radical is a highly effective oxidizing agent, reacting at a diffusion-controlled rate with most organic species, such as nucleic acids, proteins, and lipids. Furthermore, superoxide anions or a biological reductant (e.g., ascorbic acid) can reduce the resulting Fe⁺³ ion back to Fe⁺² for continued peroxide reduction, thus a problematic cycle.

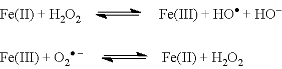

Therefore, diseases or conditions that lead to bleeding and/or an inflammatory response involve the possibility that reactive oxygen species will come in contact with Fe⁺² ions to produce highly reactive and damaging hydroxyl radicals. That is, the iron released from red blood cells react with oxygen species produced by inflammatory cells such as neutrophils to produce hydroxyl radicals that cause cell and tissue injury. The solution, therefore, is the same for conditions of focal iron overload (e.g., closed head injury, hemorrhagic stroke, IBD) as it is for global iron overload-chelation and removal of the unmanaged iron.

Various desferrithiocin analogs, including desferrithiocin polyether analogs, have been developed that effectively chelate and remove iron from biological systems. See International PCT Applications, PCT/US99/19691, filed Aug. 31, 1999; PCT/US2003/028304, filed Sep. 9, 2003; PCT/US2006/010945, filed Mar. 22, 2006; PCT/US2008/003433, filed Mar. 14, 2008; PCT/US2010/002336, filed Aug. 25, 2010; each of which is incorporated herein by reference. Therefore, the present invention applies the use of these analogs, which have been previously only suggested for use in the treatment of global metal overload, to diseases and conditions associated with focal iron overload, such as, but not limited to, macular degeneration, stroke, IBD, closed head injury, and reperfusion injury. In certain embodiments, the desferrithiocin analog useful in the present invention is of Formula (I):

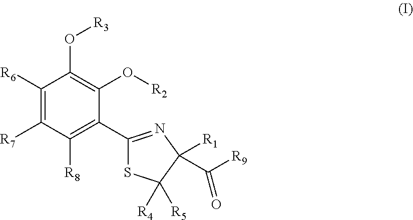

wherein R₁-R₉ are as defined here. In certain embodiments, desferrithiocin analogs with a polyether moiety at the 3′-position of the phenyl ring are used in the present invention (i.e., R₃ is a polyether moiety). Such analogs have been found to be useful in treating diseases or conditions associated with focal iron overload, for example, where iron has been introduced into an organ, tissue, or space by bleeding or through an inflammatory response. In certain embodiments, such analogs have been found in the cerebral spinal fluid and therefore may be useful in treating neurological diseases such as closed head injury or stroke. In certain embodiments, such analogs have been found to penetrate into the eye and may be useful in treating ophthalmologic diseases such as macular degeneration. All of these diseases are associated with free radical damage resulting from unmanaged or free iron in the respective tissue or organ. Therefore, the chelation and removal of the free iron in these tissues and organs would be effective in preventing or treating each of these diseases.

Based on this recognition the present invention provides methods of treating and preventing diseases and conditions associated with focal iron overload and pharmaceutical compositions for use in treating such diseases and conditions. The invention provides new uses for previously known compounds in the treatment of diseases and conditions associated with focal iron overload. The invention also provides kits including compounds and compositions found useful in treatment of such disease and conditions.

In one aspect, the invention provides methods of preventing or treating macular degeneration by administering to a subject an effective amount of a compound of Formula (I) to prevent or treat macular degeneration. Compounds of Formula (I) have been found to get into the eye and chelate and remove iron that is thought to contribute to the generation of reactive oxygen species in the eye that cause biological injury. Such reactive oxygen species are particularly detrimental in the retina of the eye. The present invention also provides pharmaceutical compositions suitable for ocular administration and uses of the compounds of Formula (I) and compositions thereof for the treatment of macular degeneration. In certain embodiments, the pharmaceutical composition for ocular administration is in the form of an eye drop. The compound of Formula (I) or a composition thereof may also be administered systemically for the treatment of macular degeneration.

In another aspect, the invention provides methods of removing iron from tissues or organs that have been bled into or otherwise have focal iron overload. For example, methods for the treatment of head injury, including closed head injury, are provided. Closed head injuries that may be treated by the inventive methods and compositions may result from any number of causes including falls, blasts, sports injuries, accidents including vehicular accidents, and assaults. In certain embodiments, the inventive method comprises administering to a subject an effective amount of a compound of Formula (I) to sequester iron resulting from a hemorrhage or vascular compromise in the head. In certain embodiments, the subject has suffered from a closed head injury. In other embodiments, the subject has suffered from or is at risk of suffering from a stroke (e.g., a hemorrhagic stroke). The present invention also provides pharmaceutical compositions for the treatment of head injury and uses of the compounds of Formula (I) and compositions thereof for the treatment of head injury. In certain embodiments for the treatment of head injury, the compound of Formula (I) or a composition thereof is administered systemically (e.g., orally or parenterally).

In another aspect, the invention provides methods for the treatment of stroke, particularly hemorrhagic stroke. Such methods include administering a compound of Formula (I) to a subject at risk of having a stroke or having had a stroke. In certain embodiments, the method comprises administering to a subject who has had or is at risk of having a stroke an effective amount of a compound of Formula (I). Without wishing to be bound by a particular theory, the administered compound is thought to sequester iron resulting from hemorrhage or vascular compromise thereby preventing or at least lessening tissue damage caused by reactive oxygen species. Such reactive oxygen species may be generated by free iron ions resulting from the bleed in the brain. In certain embodiments, the subject has suffered from a hemorrhagic stroke. In other embodiments, the subject is at risk of having a hemorrhagic stroke. The present invention also provides pharmaceutical compositions for the treatment of stroke and uses of the compounds of Formula (I) and compositions thereof for the treatment of stroke. In certain embodiments for the treatment of stroke, the compound of Formula (I) or a composition thereof is administered systemically (e.g., orally or parenterally).

In another aspect, the invention provides methods for preventing or lessening reperfusion injury. Reperfusion injury is caused by reactive oxygen species that are generated when the blood supply returns to a tissue after a period of ischemia. Compounds of Formula (I) or compositions thereof are administered to a subject at risk of reperfusion injury to prevent the formation of reactive oxygen species or inactivate free radical species. Ischemia may result from a number of causes including stroke, myocardial infarction, infarction of other tissues or organs, surgery (e.g., cardiac surgery), and organ donation and transplantation. The present invention also provides pharmaceutical compositions for the prevention and treatment of reperfusion injury as well as the uses of compounds of Formula (I) and compositions thereof for the prevention and treatment of reperfusion injury. The compounds of Formula (I) or compositions thereof may be administered locally or systemically in the prevention or treatment of reperfusion injury.

In yet another aspect, the present invention provides methods of treating irritable bowel disease (IBD). Reactive oxygen species have been found important in the pathogenesis of IBD; therefore, as described above for the treatment of reperfusion injury, any compound, composition, or treatment that chelates and removes iron and/or quenches free radicals would be useful in the treatment of IBD. In certain embodiments, the method comprises administering to a subject an effective amount of a compound of Formula (I) or a composition thereof to treat IBD. The present invention also provides pharmaceutical compositions for the treatment of IBD and the uses of the compounds of Formula (I) and compositions thereof for the treatment of IBD. The compounds of Formula (I) or compositions thereof may be administered locally (e.g., rectally) or systemically in the treatment of IBD.

The present invention also provides kits with the compound of Formula (I) or compositions thereof for use in the treatment of macular degeneration, head injury (e.g., closed head injury), stroke (e.g., hemorrhagic stroke), reperfusion injury, and IBD. Such kits may include one or more unit dosage forms of the compound or composition to be administered to a subject. In certain embodiments, the kit may include enough unit dosage forms for a course of treatment or for a particular time period (e.g., a week, 10 days, 14 days, a month). The kits may also include packaging information describing the use or prescribing information for the subject or a health care professional. Such information may be required by a regulatory agency such as the U.S. Food and Drug Administration (FDA). The kit may also optionally include a device for administration of the compound or composition, for example, a dropper for ocular administration or a syringe for parenteral administration.

The details of one or more embodiments of the invention are set forth in the accompanying Detailed Description, Examples, Claims, and Figures. Other features, objects, and advantages of the invention will be apparent from the description and claims.

The references, web pages, scientific journal articles, patent applications, and issued patents cited in this application are incorporated herein by reference.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

The compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention.

Where an isomer/enantiomer is preferred, it may, in some embodiments, be provided substantially free of the corresponding enantiomer, and may also be referred to as “optically enriched” or “enantiomerically enriched.” “Optically enriched” and “enantiomerically enriched,” as used herein, means that a provided compound is made up of a significantly greater proportion of one enantiomer. In certain embodiments, a compound of the present invention is made up of at least about 70% by weight of a preferred enantiomer. In certain embodiments, a compound of the present invention is made up of at least about 80% by weight of a preferred enantiomer. In certain embodiments, a compound of the present invention is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); Wilen, Tables of Resolving Agents and Optical Resolutions p. 268 (E. L. Eliel, Ed., Univ. of Notre Dame Press, Notre Dame, Ind. 1972).

Unless otherwise stated, structures depicted herein are also meant to include compounds that differ only in the presence of one or more isotopically enriched atoms. For example, compounds having the depicted structures that differ only in the replacement of hydrogen by deuterium or tritium, or the replacement of a carbon by ¹³C or ¹⁴C are within the scope of this invention. Such compounds are useful, for example, as analytical tools, as probes in biological assays, or as therapeutic agents in accordance with the present invention.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

The terms “purified,” “substantially purified,” and “isolated” as used herein refer to a compound useful in the present invention being free of other, dissimilar compounds with which the compound is normally associated in its natural state, so that the compound comprises at least 0.5%, 1%, 5%, 10%, 20%, 50%, 75%, 80%, 85%, 90%, 95%, 96%, 97%, 98%, 99%, 99.5%, 99.9% of the mass, by weight, of a given sample or composition. In one embodiment, these terms refer to the compound comprising at least 95%, 98%, 99%, or 99.9% of the mass, by weight, of a given sample or composition.

The term “acyl,” as used herein, refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “acyloxy” refers to a “substituted hydroxyl” of the formula (—ORⁱ), wherein Rⁱ is an optionally substituted acyl group, as defined herein, and the oxygen moiety is directly attached to the parent molecule.

The term “aliphatic,” as used herein, includes both saturated and unsaturated, nonaromatic, straight chain (i.e., unbranched), branched, acyclic, and cyclic (i.e., carbocyclic) hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “aliphatic” is used to indicate those aliphatic groups (cyclic, acyclic, substituted, unsubstituted, branched or unbranched) having 1-20 carbon atoms. Aliphatic group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “alkyl,” as used herein, refers to saturated, straight- or branched-chain hydrocarbon radicals derived from a hydrocarbon moiety containing between one and twenty carbon atoms by removal of a single hydrogen atom. In some embodiments, the alkyl group employed in the invention contains 1-20 carbon atoms. In another embodiment, the alkyl group employed contains 1-15 carbon atoms. In another embodiment, the alkyl group employed contains 1-10 carbon atoms. In another embodiment, the alkyl group employed contains 1-8 carbon atoms. In another embodiment, the alkyl group employed contains 1-5 carbon atoms. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more substituents. Alkyl group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “alkenyl,” as used herein, denotes a monovalent group derived from a straight- or branched-chain hydrocarbon moiety having at least one carbon-carbon double bond by the removal of a single hydrogen atom. In certain embodiments, the alkenyl group employed in the invention contains 2-20 carbon atoms. In some embodiments, the alkenyl group employed in the invention contains 2-15 carbon atoms. In another embodiment, the alkenyl group employed contains 2-10 carbon atoms. In still other embodiments, the alkenyl group contains 2-8 carbon atoms. In yet other embodiments, the alkenyl group contains 2-5 carbons. Alkenyl groups include, for example, ethenyl, propenyl, butenyl, 1-methyl-2-buten-1-yl, and the like, which may bear one or more substituents. Alkenyl group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “alkynyl,” as used herein, refers to a monovalent group derived from a straight- or branched-chain hydrocarbon having at least one carbon-carbon triple bond by the removal of a single hydrogen atom. In certain embodiments, the alkynyl group employed in the invention contains 2-20 carbon atoms. In some embodiments, the alkynyl group employed in the invention contains 2-15 carbon atoms. In another embodiment, the alkynyl group employed contains 2-10 carbon atoms. In still other embodiments, the alkynyl group contains 2-8 carbon atoms. In still other embodiments, the alkynyl group contains 2-5 carbon atoms. Representative alkynyl groups include, but are not limited to, ethynyl, 2-propynyl (propargyl), 1-propynyl, and the like, which may bear one or more substituents. Alkynyl group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₉ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X is a counterion.

The term “amino,” as used herein, refers to a group of the formula (—NH₂). A “substituted amino” refers either to a mono-substituted amine (—NHR^{h}) of a disubstituted amine (—NR^{h}₂), wherein the R^{h} substituent is any substituent as described herein that results in the formation of a stable moiety (e.g., a suitable amino protecting group; aliphatic, alkyl, alkenyl, alkynyl, heteroaliphatic, heterocyclic, aryl, heteroaryl, acyl, amino, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted). In certain embodiments, the R^{h} substituents of the disubstituted amino group(—NR^{h}₂) form a 5- to 6-membered heterocyclic ring.

The term “alkoxy” refers to a “substituted hydroxyl” of the formula (—ORⁱ), wherein Rⁱ is an optionally substituted alkyl group as defined herein, and the oxygen moiety is directly attached to the parent molecule.

The term “alkylthioxy” refers to a “substituted thiol” of the formula (—SR^{r}), wherein R^{r} is an optionally substituted alkyl group as defined herein, and the sulfur moiety is directly attached to the parent molecule.

The term “alkylamino” refers to a “substituted amino” of the formula (—NR^{h}₂), wherein R^{h} is, independently, a hydrogen or an optionally substituted alkyl group as defined herein, and the nitrogen moiety is directly attached to the parent molecule.

The term “aryl,” as used herein, refer to stable aromatic mono- or polycyclic ring system having 3-20 ring atoms, of which all the ring atoms are carbon, and which may be substituted or unsubstituted. In certain embodiments of the present invention, “aryl” refers to a mono, bi, or tricyclic C₄-C₂₀ aromatic ring system having one, two, or three aromatic rings which include, but not limited to, phenyl, biphenyl, naphthyl, and the like, which may bear one or more substituents. Aryl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “arylalkyl,” as used herein, refers to an aryl substituted alkyl group, wherein the terms “aryl” and “alkyl” are defined herein, and wherein the aryl group is attached to the alkyl group, which in turn is attached to the parent molecule. An exemplary arylalkyl group is benzyl.

The term “aryloxy” refers to a “substituted hydroxyl” of the formula (—ORⁱ), wherein Rⁱ is an optionally substituted aryl group as defined herein, and the oxygen moiety is directly attached to the parent molecule.

The term “arylamino,” refers to a “substituted amino” of the formula (—NR^{h}₂), wherein R^{h} is, independently, a hydrogen or an optionally substituted aryl group as defined herein, and the nitrogen moiety is directly attached to the parent molecule.

The term “arylthioxy” refers to a “substituted thiol” of the formula (—SR^{r}), wherein R^{r} is an optionally substituted aryl group as defined herein, and the sulfur moiety is directly attached to the parent molecule.

The term “azido,” as used herein, refers to a group of the formula (—N₃).

The term “cyano,” as used herein, refers to a group of the formula (—CN).

The terms “halo” and “halogen” as used herein refer to an atom selected from fluorine (fluoro, —F), chlorine (chloro, —Cl), bromine (bromo, —Br), and iodine (iodo, —I).

The term “heteroaliphatic,” as used herein, refers to an aliphatic moiety, as defined herein, which includes both saturated and unsaturated, nonaromatic, straight chain (i.e., unbranched), branched, acyclic, cyclic (i.e., heterocyclic), or polycyclic hydrocarbons, which are optionally substituted with one or more functional groups, and that contain one or more oxygen, sulfur, nitrogen, phosphorus, or silicon atoms, e.g., in place of carbon atoms. In certain embodiments, heteroaliphatic moieties are substituted by independent replacement of one or more of the hydrogen atoms thereon with one or more substituents. As will be appreciated by one of ordinary skill in the art, “heteroaliphatic” is intended herein to include, but is not limited to, heteroalkyl, heteroalkenyl, heteroalkynyl, heterocycloalkyl, heterocycloalkenyl, and heterocycloalkynyl moieties. Thus, the term “heteroaliphatic” includes the terms “heteroalkyl,” “heteroalkenyl”, “heteroalkynyl”, and the like. Furthermore, as used herein, the terms “heteroalkyl”, “heteroalkenyl”, “heteroalkynyl”, and the like encompass both substituted and unsubstituted groups. In certain embodiments, as used herein, “heteroaliphatic” is used to indicate those heteroaliphatic groups (cyclic, acyclic, substituted, unsubstituted, branched or unbranched) having 1-20 carbon atoms. Heteroaliphatic group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “heteroalkyl,” as used herein, refers to an alkyl moiety, as defined herein, which contain one or more oxygen, sulfur, nitrogen, phosphorus, or silicon atoms, e.g., in place of carbon atoms.

The term “heteroalkenyl,” as used herein, refers to an alkenyl moiety, as defined herein, which contain one or more oxygen, sulfur, nitrogen, phosphorus, or silicon atoms, e.g., in place of carbon atoms.

The term “heteroalkynyl,” as used herein, refers to an alkynyl moiety, as defined herein, which contain one or more oxygen, sulfur, nitrogen, phosphorus, or silicon atoms, e.g., in place of carbon atoms.

The term “heteroalkylamino” refers to a “substituted amino” of the formula (—NR^{h}₂), wherein R^{h} is, independently, a hydrogen or an optionally substituted heteroalkyl group, as defined herein, and the nitrogen moiety is directly attached to the parent molecule.

The term “heteroalkyloxy” refers to a “substituted hydroxyl” of the formula (—ORⁱ), wherein Rⁱ is an optionally substituted heteroalkyl group, as defined herein, and the oxygen moiety is directly attached to the parent molecule.

The term “heteroalkylthioxy” refers to a “substituted thiol” of the formula (—SR^{r}), wherein R^{r} is an optionally substituted heteroalkyl group, as defined herein, and the sulfur moiety is directly attached to the parent molecule.

The term “heterocyclic,” “heterocycles,” or “heterocyclyl,” as used herein, refers to a cyclic heteroaliphatic group. A heterocyclic group refers to a non-aromatic, partially unsaturated or fully saturated, 3- to 12-membered ring system, which includes single rings of 3 to 8 atoms in size, and bi- and tri-cyclic ring systems which may include aromatic five- or six-membered aryl or heteroaryl groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocyclic refers to a non-aromatic 5-, 6-, or 7-membered ring or polycyclic group wherein at least one ring atom is a heteroatom selected from O, S, and N (wherein the nitrogen and sulfur heteroatoms may be optionally oxidized), and the remaining ring atoms are carbon, the radical being joined to the rest of the molecule via any of the ring atoms. Heterocyclyl groups include, but are not limited to, a bi- or tri-cyclic group, comprising fused five, six, or seven-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Exemplary heterocycles include azacyclopropanyl, azacyclobutanyl, 1,3-diazatidinyl, piperidinyl, piperazinyl, azocanyl, thiaranyl, thietanyl, tetrahydrothiophenyl, dithiolanyl, thiacyclohexanyl, oxiranyl, oxetanyl, tetrahydrofuranyl, tetrahydropuranyl, dioxanyl, oxathiolanyl, morpholinyl, thioxanyl, tetrahydronaphthyl, and the like, which may bear one or more substituents. Substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “heteroaryl,” as used herein, refer to stable aromatic mono- or polycyclic ring system having 3-20 ring atoms, of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms. Exemplary heteroaryls include, but are not limited to pyrrolyl, pyrazolyl, imidazolyl, pyridinyl, pyrimidinyl, pyrazinyl, pyridazinyl, triazinyl, tetrazinyl, pyrrolizinyl, indolyl, quinolinyl, isoquinolinyl, benzoimidazolyl, indazolyl, quinolinyl, isoquinolinyl, quinolizinyl, cinnolinyl, quinazolynyl, phthalazinyl, naphthridinyl, quinoxalinyl, thiophenyl, thianaphthenyl, furanyl, benzofuranyl, benzothiazolyl, thiazolynyl, isothiazolyl, thiadiazolynyl, oxazolyl, isoxazolyl, oxadiaziolyl, oxadiaziolyl, and the like, which may bear one or more substituents. Heteroaryl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “heteroarylene,” as used herein, refers to a biradical derived from an heteroaryl group, as defined herein, by removal of two hydrogen atoms. Heteroarylene groups may be substituted or unsubstituted. Additionally, heteroarylene groups may be incorporated as a linker group into an alkylene, alkenylene, alkynylene, heteroalkylene, heteroalkenylene, or heteroalkynylene group, as defined herein. Heteroarylene group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “heteroarylamino” refers to a “substituted amino” of the (—NR^{h}₂), wherein R^{h} is, independently, a hydrogen or an optionally substituted heteroaryl group, as defined herein, and the nitrogen moiety is directly attached to the parent molecule.

The term “heteroaryloxy” refers to a “substituted hydroxyl” of the formula (—ORⁱ), wherein Rⁱ is an optionally substituted heteroaryl group, as defined herein, and the oxygen moiety is directly attached to the parent molecule.

The term “heteroarylthioxy” refers to a “substituted thiol” of the formula (—SR^{r}), wherein R^{r} is an optionally substituted heteroaryl group, as defined herein, and the sulfur moiety is directly attached to the parent molecule.

The term “hydroxy,” or “hydroxyl,” as used herein, refers to a group of the formula (—OH). A “substituted hydroxyl” refers to a group of the formula (—ORⁱ), wherein Rⁱ can be any substituent which results in a stable moiety (e.g., a suitable hydroxyl protecting group; aliphatic, alkyl, alkenyl, alkynyl, heteroaliphatic, heterocyclic, aryl, heteroaryl, acyl, nitro, alkylaryl, arylalkyl, and the like, each of which may or may not be further substituted).

The term “imino,” as used herein, refers to a group of the formula (═NR^{r}), wherein R^{r} corresponds to hydrogen or any substituent as described herein, that results in the formation of a stable moiety (for example, a suitable amino protecting group; aliphatic, alkyl, alkenyl, alkynyl, heteroaliphatic, heterocyclic, aryl, heteroaryl, acyl, amino, hydroxyl, alkylaryl, arylalkyl, and the like, each of which may or may not be further substituted). In certain embodiments, imino refers to ═NH wherein R^{r} is hydrogen.

The term “isocyano,” as used herein, refers to a group of the formula (—NC).

The term “nitro,” as used herein, refers to a group of the formula (—NO₂).

The term “oxo,” as used herein, refers to a group of the formula (═O).

The term “stable moiety,” as used herein, preferably refers to a moiety which possess stability sufficient to allow manufacture, and which maintains its integrity for a sufficient period of time to be useful for the purposes detailed herein.

A “protecting group,” as used herein, is well known in the art and include those described in detail in Greene's Protective Groups in Organic Synthesis, P. G. M. Wuts and T. W. Greene, 4^{th} edition, Wiley-Interscience, 2006, the entirety of which is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

A “suitable hydroxyl protecting group” as used herein, is well known in the art and includes those described in detail in Greene (1999). Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4′-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-naphthyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

The term “subject,” as used herein, refers to any animal. In certain embodiments, the subject is a mammal. In certain embodiments, the term “subject”, as used herein, refers to a human (e.g., a man, a woman, or a child). The human may be of either sex and may be at any stage of development. In certain embodiments, the subject has been diagnosed with the condition or disease to be treated (e.g., macular degeneration, stroke, IBD, closed head injury). In other embodiments, the subject is at risk of developing the condition or disease. In certain embodiments, the subject is an experimental animal (e.g., mouse, rat, dog, primate). The experimental animal may be genetically engineered. In certain embodiments, the subject is a domesticated animal (e.g., dog, cat, bird, horse, cow, goat, sheep).

The terms “administer,” “administering,” or “administration,” as used herein refers to implanting, absorbing, ingesting, injecting, or inhaling a compound of Formula (I) or a pharmaceutical composition thereof.

As used herein, the terms “treatment,” “treat,” and “treating” refer to reversing, alleviating, delaying the onset of, or inhibiting the progress of a disease or disorder, or one or more signs or symptoms thereof, described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example to delay or prevent recurrence.

The terms “effective amount” and “therapeutically effective amount,” as used herein, refer to the amount or concentration of an inventive compound, that, when administered to a subject, is effective to at least partially treat a condition from which the subject is suffering (e.g., chronic inflammatory disease, autoimmune disease, dry eye syndrome, fibrosis, scar formation, angiogenesis, viral infection, malaria, ischemic damage, transplant and implant rejection, neurodegenerative disease, or a cosmetic indication).

The term “focal iron overload” refers to any disease or condition that involves the accumulation of unmanaged iron in a tissue or organ. Focal iron overload typically involves less than the subject's whole body but may involve more than one organ or tissue. Unmanaged iron in any tissue or organ is typically undesired and can be the focus of the treatments of the present invention. The treatment may involve the removal of as much iron as possible from the tissue or organ or may only involve the removal of excess iron. Examples of disease and conditions associated with focal iron overload include, but are not limited to, macular degeneration, IBD, reperfusion injury, stroke including hemorrhagic stroke, and closed head injury; however, any disease or condition of focal iron overload may be treated as described herein. In certain embodiments, the term “focal iron overload” does not include diseases or conditions associated with global iron overload (e.g., global iron overload associated with chronic transfusion therapy, hereditary hemochromatosis, etc.). The treatment of focal iron overload may be systemic or local administration of an effective amount of a compound of Formula (I).

The term “reactive oxygen species” or “ROS” refers to molecules or ions formed by the incomplete reduction of oxygen. Reactive oxygen species include superoxide anion (O₂.⁻), peroxides such as hydrogen peroxide (H₂O₂), hydroxyl radical (HO.), and hypochlorous acid (HClO). These molecules are typically chemically reactive. Reactive oxygen species may be formed by any number of mechanisms (e.g. enzymatically, by ionizing radiation, by reaction oxygen with a metal). In certain embodiments, the reactive oxygen species are formed by the reduction of oxygen by an iron ion such as Fe⁺².

The term “closed head injury” refers to any injury to the head that does not penetrate the skull. Closed head injuries may result from falls, blasts, accidents including vehicular accidents, and assaults. Closed head injuries can lead to hemorrhage or brain swelling, which can result in increased intracranial pressure, which can in turn lead to permanent brain damage or even death. Various types of closed head injury include concussions, brain contusions, diffuse axonal injury, and hematomas.

The term “tautomer” refers to a particular isomer of a compound in which a hydrogen and double bond have changed position with respect to the other atoms of the molecule. For a pair of tautomers to exist there must be a mechanism for interconversion. Examples of tautomers include keto-enol forms, imine-enamine forms, amide-imino alcohol forms, amidine-aminidine forms, nitroso-oxime forms, thio ketone-enethiol forms, N-nitroso-hydroxyazo forms, nitro-aci-nitro forms, lactam-lactim forms, ketene-ynol forms, enamine-enamine forms, and pyridione-hydroxypyridine forms.

### BRIEF DESCRIPTION OF THE DRAWINGS

FIG. 1 shows representative colons from rats treated with desferrioxamine (DFO) (20 mg/kg) IV and sterile water (control) 30 minutes before the 4% acetic acid. Each colon is positioned such that the cecum is on the right and the rectum is on the left.

FIG. 2 is a bar graph showing the concentration of Deferitrin and (S)-3′-(HO)-DADFT-PE (III-A) in plasma at various time points after a 300 tmol/kg oral (po) dose of each compound in rats.

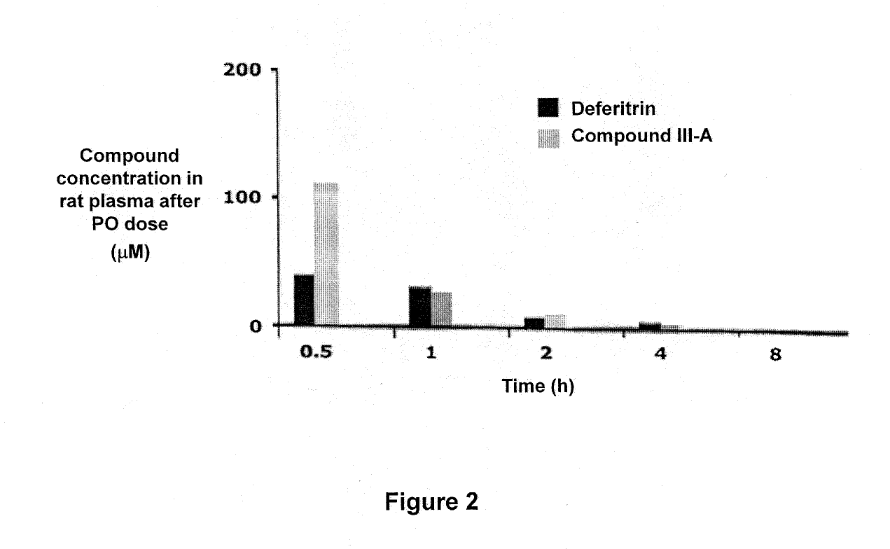

FIG. 3 is a bar graph showing the concentration of Deferitrin, (S)-3′-(HO)-DADFT-PE (III-A), and (S)-3′-(HO)-DADFT-norPE (II-A) in plasma at various time points after a 300 μmol/kg subcutaneous (sc) dose of each compound in rats.

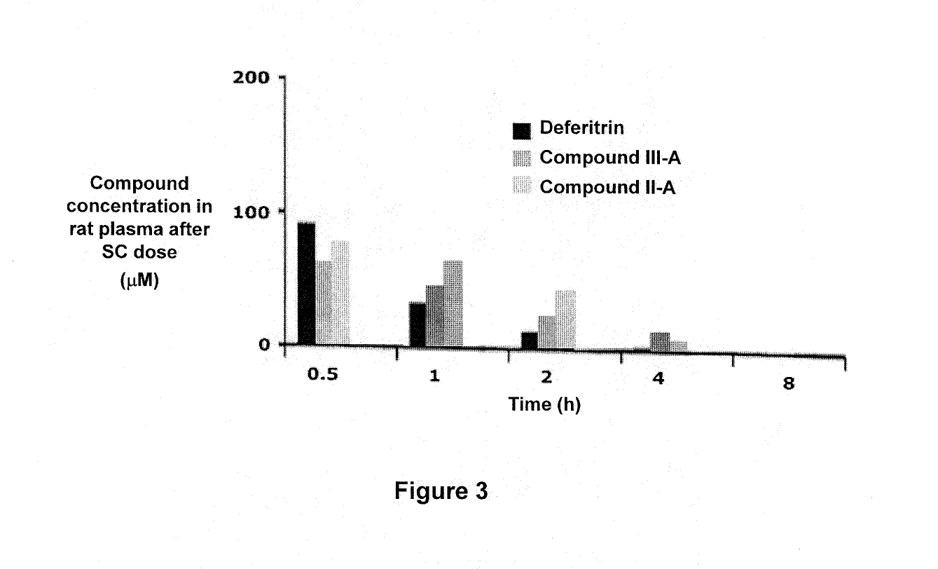

### DETAILED DESCRIPTION OF CERTAIN EMBODIMENTS OF THE INVENTION

Various desferrithiocin analogs have been described for use in the treatment of global iron overload resulting from transfusion therapy, high-iron diet, acute iron ingestion, or malabsorption. Such analogs have now been discovered to be useful in treating or preventing diseases and conditions associated with focal iron overload, where the local concentration of iron in a particular tissue or organ contributes to the pathological process. For instance, the unmanaged Fe⁺² ions in a tissue or organ may result in the production of hydroxyl radicals or other reactive oxygen species that lead to tissue or cell damage. Therefore, desferrithiocin analogs of Formula (I), particularly those with a polyether moiety at the 3′-position of the phenyl ring, are expected to be useful in the treatment of macular degeneration, closed head injury, reperfusion injury, and stroke. Without wishing to be bound by any particular theory, the compounds of Formula (I) are thought to chelate iron and prevent it from participating in the generation of reactive oxygen species. The compounds of Formula (I) may also act as free radical scavenger thereby limiting the damage of reactive oxygen species or other radicals. The invention, therefore, provides methods of treating and preventing disease and conditions associated with focal iron overload, as well as pharmaceutical compositions and kits useful in the inventive methods.

### Useful Compounds

Desferrithiocin analogs of Formula (I) are expected to be useful in preventing and treating diseases and conditions associated with iron overload, particularly focal iron overload. Such analogs have been previously described in International PCT Applications, PCT/US2008/003433, filed Mar. 14, 2008, and U.S. patent application, U.S. Ser. No. 12/450,194, filed Dec. 14, 2009; each of which is incorporated herein by reference. Compounds with a polyether moiety at the 3′-position of the phenyl ring are expected to be particularly useful in the methods and compositions of the present invention.

In certain embodiments, compounds useful in the present invention are of Formula (I):

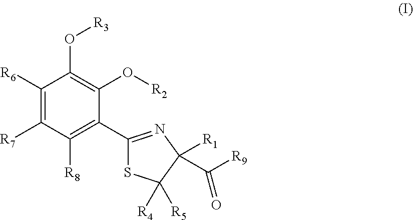

wherein:

R₁, R₄, and R₅ are each independently hydrogen, halogen, alkyl, or —OR₁₂;

R₂ is hydrogen, alkyl, or acyl;

R₃ is hydrogen, alkyl, or —[(CH₂)ₙ—O]ₓ—[(CH₂)ₙ—O]_{y}—R′;

R₆, R₇, and R₈ are each independently hydrogen, alkyl, arylalkyl, or —OR₁₀;

R₉ is —OR₁₁ or —SR₁₁;

R₁₀ is hydrogen, alkyl, or acyl;

R₁₁ is hydrogen or alkyl;

R₁₂ is hydrogen or alkyl;

R′ is alkyl;

each occurrence of n is independently an integer from 1 to 8, inclusive;

x is an integer from 1 to 8, inclusive; and

y is an integer from 0 to 8, inclusive;

or a pharmaceutically acceptable salt, tautomer, solvate, hydrate, or polymorph thereof.

In compounds of Formula (I), R₁ is hydrogen, halogen, alkyl, or —OR₁₂. In certain embodiments, R₁ is hydrogen. In certain embodiments, R₁ is halogen. In certain embodiments, R₁ is C₁-C₆ alkyl. In certain embodiments, R₁ is methyl. In certain embodiments, R₁ is ethyl. In certain embodiments, R₁ is propyl. In certain embodiments, R₁ is —OH. In certain embodiments, R₁ is —OCH₃.

In compounds of Formula (I), R₂ is hydrogen, alkyl, or acyl. In certain embodiments, R₂ is hydrogen. In certain embodiments, R₂ is C₁-C₆ alkyl. In certain embodiments, R₂ is methyl. In certain embodiments, R₂ is ethyl. In certain embodiments, R₂ is propyl. In certain embodiments, R₂ is acyl. In certain embodiments, R₂ is acetyl.

In compounds of Formula (I), R₃ is hydrogen, alkyl, or —[(CH₂)ₙ—O]ₓ—[(CH₂)ₙ—O]_{y}—R′. In certain embodiments, R₃ is hydrogen. In certain embodiments, R₃ is C₁-C₆ alkyl. In certain embodiments, R₃ is methyl. In certain embodiments, R₃ is ethyl. In certain embodiments, R₃ is propyl. In certain embodiments, R₃ is —[(CH₂)ₙ—O]ₓ—[(CH₂)ₙ—O]_{y}—R′. In certain embodiments, R₃ is —[(CH₂)₂—O]—CH₃. In certain embodiments, R₃ is —[(CH₂)₂—O]₂—CH₃. In certain embodiments, R₃ is —[(CH₂)₂—O]₃—CH₃. In certain embodiments, R₃ is —[(CH₂)₂—O]₄—CH₃. In certain embodiments, R₃ is —[(CH₂)₂—O]₅—CH₃.

In compounds of Formula (I), n is an integer from 1 to 8, inclusive. In certain embodiments, n is 2. In certain embodiments, n is 3. In certain embodiments, n is 4. In certain embodiments, n is 5.

In compounds of Formula (I), x is an integer from 1 to 8, inclusive. In certain embodiments, x is 1. In certain embodiments, x is 2. In certain embodiments, x is 3. In certain embodiments, x is 4. In certain embodiments, x is 5.

In compounds of Formula (I), y is an integer from 0 to 8, inclusive. In certain embodiments, y is 0. In certain embodiments, y is 1. In certain embodiments, y is 2. In certain embodiments, y is 3. In certain embodiments, y is 4. In certain embodiments, y is 5.

In compounds of Formula (I), R′ is alkyl. In certain embodiments, R′ is C₁-C₆ alkyl. In certain embodiments, R′ is methyl. In certain embodiments, R′ is ethyl. In certain embodiments, R′ is propyl. In certain embodiments, R′ is —CF₃.

In compounds of Formula (I), R₄ is hydrogen, halogen, alkyl, or —OR₁₂. In certain embodiments, R₄ is hydrogen. In certain embodiments, R₄ is halogen. In certain embodiments, R₄ is C₁-C₆ alkyl. In certain embodiments, R₄ is methyl. In certain embodiments, R₄ is ethyl. In certain embodiments, R₄ is propyl. In certain embodiments, R₄ is —OH. In certain embodiments, R₄ is —OCH₃.

In compounds of Formula (I), R₅ is hydrogen, halogen, alkyl, or —OR₁₂. In certain embodiments, R₅ is hydrogen. In certain embodiments, R₅ is halogen. In certain embodiments, R₅ is C₁-C₆ alkyl. In certain embodiments, R₅ is methyl. In certain embodiments, R₅ is ethyl. In certain embodiments, R₅ is propyl. In certain embodiments, R₅ is —OH. In certain embodiments, R₅ is —OCH₃.

In certain embodiments, both R₄ and R₅ are hydrogen. In certain embodiments, at least one of R₄ and R₅ is hydrogen.

In compounds of Formula (I), R₆ is each independently hydrogen, alkyl, arylalkyl, or —OR₁₀. In certain embodiments, R₆ is hydrogen. In certain embodiments, R₆ is C₁-C₆ alkyl. In certain embodiments, R₆ is methyl. In certain embodiments, R₆ is ethyl. In certain embodiments, R₆ is propyl. In certain embodiments, R₆ is —OH. In certain embodiments, R₆ is —OCH₃.

In compounds of Formula (I), R₇ is each independently hydrogen, alkyl, arylalkyl, or —OR₁₀. In certain embodiments, R₇ is hydrogen. In certain embodiments, R₇ is C₁-C₆ alkyl. In certain embodiments, R₇ is methyl. In certain embodiments, R₇ is ethyl. In certain embodiments, R₇ is propyl. In certain embodiments, R₇ is —OH. In certain embodiments, R₇ is —OCH₃.

In compounds of Formula (I), R₈ is each independently hydrogen, alkyl, arylalkyl, or —OR₁₀. In certain embodiments, R₈ is hydrogen. In certain embodiments, R₈ is C₁-C₆ alkyl. In certain embodiments, R₈ is methyl. In certain embodiments, R₈ is ethyl. In certain embodiments, R₈ is propyl. In certain embodiments, R₈ is —OH. In certain embodiments, R₈ is —OCH₃.

In certain embodiments, R₆, R₇, and R₈ are all hydrogen. In certain embodiments, at least one of R₆, R₇, and R₈ is hydrogen. In certain embodiments, at least two of R₆, R₇, and R₈ are hydrogen.

In compounds of Formula (I), R₉ is —OR₁₁ or —SR₁₁, wherein R₁₁ is hydrogen or alkyl. In certain embodiments, R₉ is —OH. In certain embodiments, R₉ is —OCH₃. In certain embodiments, R₉ is —OCH₂CH₃. In certain embodiments, R₉ is —OCH₂CH₂CH₃. In certain embodiments, R₉ is —OCH(CH₃)₂. In certain embodiments, R₉ is —SCH₃. In certain embodiments, R₉ is —SCH₂CH₃. In certain embodiments, R₉ is —SCH₂CH₂CH₃. In certain embodiments, R₉ is —SCH(CH₃)₂. In certain embodiments, R₉ is —SCH₂CH(CH₃)₂. In certain embodiments, R₁₁ is hydrogen. In certain embodiments, R₁₁ is C₁-C₆ alkyl.

In certain embodiments, R₄ is hydrogen, R₅ is hydrogen, and R₁ is methyl.

In certain embodiments, R₆ is hydrogen, R₇ is hydrogen, R₈ is hydrogen, R₄ is hydrogen, R₅ is hydrogen, and R₁ is methyl.

In certain embodiments, R₂ is hydrogen, R₆ is hydrogen, R₇ is hydrogen, R₈ is hydrogen, R₄ is hydrogen, R₅ is hydrogen, and R₁ is methyl.

In certain embodiments, R₂ is hydrogen, R₆ is hydrogen, R₇ is hydrogen, R₈ is hydrogen, R₄ is hydrogen, R₅ is hydrogen, R₁ is methyl, and R₉ is —OH.

The compounds of Formula (I) may be provided in various salts forms. In certain embodiments, when R₉ is —OH, the compound may be provided as a carboxylate salt with a positively charged counterion. In certain embodiments, the counterion is betaine, choline hydroxide, diethanolamine, diethylamine, ethanolamine, hydroxyethylmorpholine, 4-(2-hydroxyethyl morpholine), 1-(2-hydroxyethyl pyrrolidine), 1-(2-hydroxyethyl)-piperidine, 1,2-EDSA, HCl, H₂SO₄, MSA, p-TSA, hydroxyethyl pyrroldine, imidazone, lysine (e.g., L-lysine), arginine (e.g., L-arginine), histidine (e.g., L-histidine)N-methyl-D-glucamine (NMG), N,N′-dibenzyl-ethylenediamine, N,N′-diethyl-ethanolamine, triethanolamine, tromethamine, calcium (e.g., Ca(OH)₂), magnesium (e.g., Mg(OH)₂, magnesium acetate), potassium (e.g., KOH, potassium 2-ethylhexanoate), sodium (e.g., NaOH, sodium acetate, sodium 2-ethylhexanoate), zinc (e.g., Zn(OH)₂, zinc acetate), Zn(OH)₂/Mg(OH)₂, EDA, or piperazine. In certain embodiments, the counterion is lysine. In certain embodiments, the counterion is N-methyl-D-glucamine (NMG). In certain embodiments, the counterion is tromethamine. In certain embodiments, the counterion is calcium. In certain embodiments, the counterion is magnesium. In certain embodiments, the counterion is potassium. In certain embodiments, the counterion is sodium, In certain embodiments, the counterion is zinc. In certain embodiments, the counterion is piperazine. In certain embodiments, the counterion is MgOH⁺. In certain embodiments, the counterion is ZnOH⁺.

In certain embodiments, a polymorph of a salt of a compound of Formula (I) is provided. In certain embodiments, a polymorph of a magnesium salt of a compound of Formula (I) is provided. In certain embodiments, a polymorph of a MgOH⁺ salt of a compound of Formula (I) is provided. In certain embodiments, a polymorph of a salt of a carboxylate compound of Formula (I), wherein R₉ is —OH, is provided. In certain embodiments, a polymorph of a magnesium salt of a carboxylate compound of Formula (I), wherein R₉ is —OH, is provided. In certain embodiments, a polymorph of a MgOH⁺ salt of a carboxylate compound of Formula (I), wherein R₉ is —OH, is provided.

In certain embodiments, the compound of Formula (I) is of Formula (II):

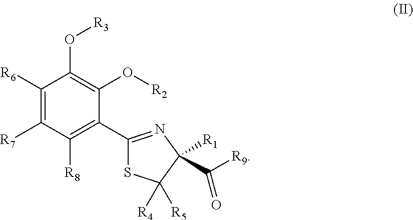

In certain embodiments, the compound of Formula (I) is of Formula (II-A):

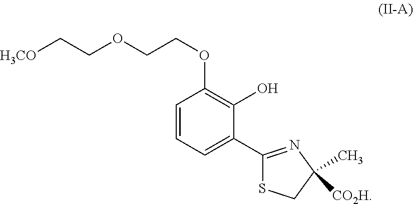 [SMILES: COCCOCCOc1cccc(C2=N[C@@](C)(C(=O)O)CS2)c1O]

In certain embodiments, a salt of a compound of Formula (II-A) is provided. In certain embodiments, a magnesium hydroxide salt of Formula (II-A) is provided as shown in Formula (II-A′):

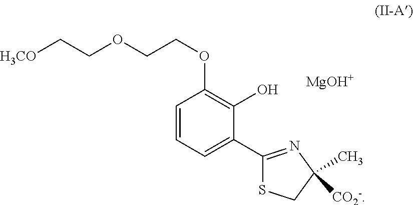

In certain embodiments, the compound of Formula (I) is of Formula (II-B):

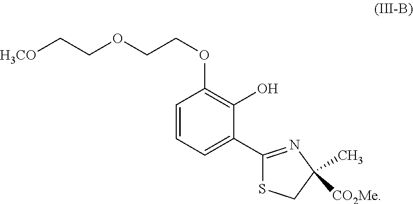 [SMILES: COCCOCCOc1cccc(C2=N[C@@](C)(C(=O)OC)CS2)c1O]

In certain embodiments, the compound of Formula (I) is of Formula (II-C):

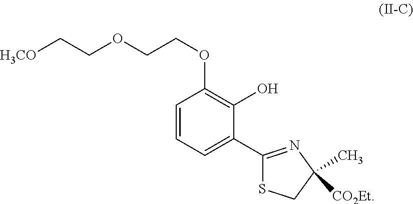 [SMILES: CCOC(=O)[C@@]1(C)CSC(c2cccc(OCCOCCOC)c2O)=N1]

In certain embodiments, the compound of Formula (I) is of Formula (III-A):

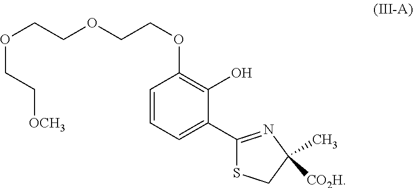 [SMILES: COCCOCCOCCOc1cccc(C2=N[C@@](C)(C(=O)O)CS2)c1O]

In certain embodiments, a salt of a compound of Formula (III-A) is provided. In certain embodiments, a magnesium hydroxide salt of Formula (III-A) is provided as shown in Formula (III-A′):

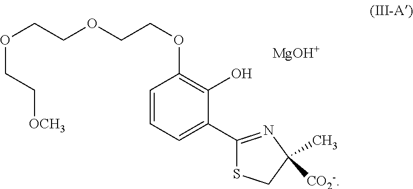

In certain embodiments, the compound is a polymorph of a salt of Formula (III-A′). In certain embodiments, the compound is a polymorph A, B, or C of a salt of Formula (III-A′) as described in U.S. Pat. No. 8,063,227, which is incorporated herein by reference. In certain embodiments, the compound is magnesium (S)-2-(2-hydroxy-3-(2-(2-(2-methoxyethoxy)ethoxy)ethoxy)phenyl-4-methyl-4,5-dihydrothiazole-4-carboxylate hydroxide, Polymorph Form A.

In certain embodiments, the compound of Formula (I) is of Formula (III-B):

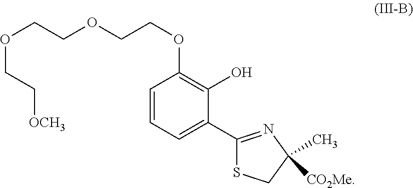 [SMILES: COCCOCCOCCOc1cccc(C2=N[C@@](C)(C(=O)OC)CS2)c1O]

In certain embodiments, the compound of Formula (I) is of Formula (I) is of Formula (III-C):

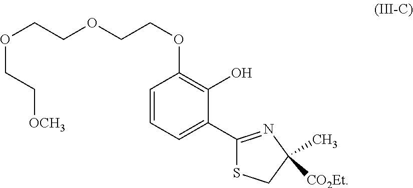 [SMILES: CCOC(=O)[C@@]1(C)CSC(c2cccc(OCCOCCOCCOC)c2O)=N1]

In certain embodiments, the compound of Formula (I) is of Formula (V-A):

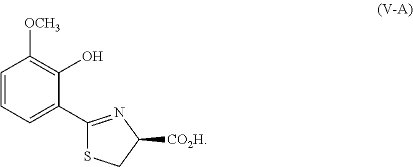 [SMILES: COc1cccc(C2=N[C@@H](C(=O)O)CS2)c1O]

In certain embodiments, the compound of Formula (I) is of Formula (V-B):

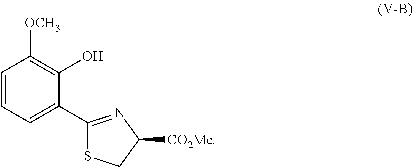 [SMILES: COC(=O)[C@H]1CSC(c2cccc(OC)c2O)=N1]

In certain embodiments, the compound of Formula (I) is of Formula (V-C):

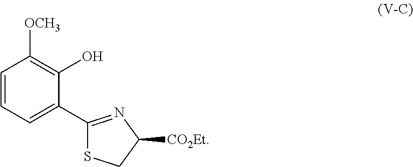 [SMILES: CCOC(=O)[C@H]1CSC(c2cccc(OC)c2O)=N1]

### Treatment of Macular Degeneration

In one aspect, the invention provides methods and pharmaceutical compositions for the treatment of macular degeneration. Without wishing to be bound by a particular theory, the compounds of Formula (I) are expected to get into the eye. As shown in U.S. Provisional Patent Application, filed Dec. 16, 2011, entitled “Uses of 4′-Desferrithiocin Analogs” by Bergeron, 4′-desferritioncin analogs have been shown to get into the eyes of rats after systemic administration. The compounds of Formula (I) would therefore be expected to chelate and remove iron from the eye thereby preventing Fe⁺² from forming and generating reactive oxygen species. The local accumulation of iron is thought to contribute to macular degeneration. Therefore, the removal of iron from the eye (including the retina) can prevent and treat macular degeneration.

In the treatment of macular degeneration, the compound of Formula (I) or a pharmaceutical composition thereof may be administered systemically or ocularly. In certain embodiments, the compound or composition is administered orally. In other embodiments, the compound or composition is administered to the eye using eyedrops or an ointment suitable for ocular administration.

The subject being treated for macular degeneration may be any type of animal. In certain embodiments, the animal is a mammal. In certain embodiments, the animal is a human. In certain embodiments, the animal is a domesticated animal (e.g., dog, cat, pig, cow). In certain embodiments, the animal is a research animal (e.g., mice, rat, dog, primate).

The exact amount of the compound of Formula (I) required to treat or prevent macular degeneration will vary from subject to subject, depending on the species, age, and general condition of the subject, the particular agent being administered, its mode of administration, and the like. The compound is preferably formulated in a dosage unit form for ease of administration and uniformity of dosage. It will be understood, however, that the total daily dosage will be decided by a physician within the scope of sound medical judgment. The specific therapeutically effective dose level for any particular subject will depend upon a variety of factors including the severity of the macular degeneration; the specific compound be administered; the age, body weight, general health, sex, and diet of the subject; the time of administration, route of administration, and rate of excretion of the particular compound being administered; the duration of the treatment; drugs used in combination or coincidental with the particular compound being administered; and like factors well known in the medical arts. In certain embodiments, the daily dosage of the compound of Formula (I) for the treatment of macular degeneration in a subject may range from 0.01 mg/kg to 200 mg/kg per unit dosage. In certain embodiments, the daily dosage ranges from 0.1 mg/kg to 100 mg/kg. In certain embodiments, the daily dosage ranges from 0.1 mg/kg to 50 mg/kg. In certain embodiments, the daily dosage ranges from 0.1 mg/kg to 20 mg/kg. In certain embodiments, the daily dosage ranges from 0.1 mg/kg to 10 mg/kg. In certain embodiments, the daily dosage ranges from 0.1 mg/kg to 1 mg/kg. In certain embodiments, the compound or a composition thereof may be administered once a day to multiple times per day. In certain embodiments, a fraction of the daily dose is administered once, twice, three times, or four times daily. In other embodiments, the compound of a composition thereof is administered every other day, every third day, every week, every other week, or every month.

### Treatment of Head Injury

The compounds of Formula (I) and pharmaceutical compositions thereof are expected to be useful in the treatment of head injury, particularly those involving bleeding into the brain or other parts of the central nervous system. Without wishing to be bound by any particular theory, the compounds of Formula (I) are thought to chelate the iron from red blood cells the blood resulting from the head injury, thereby preventing iron ions from generating reactive oxygen species. In the case of head injury resulting in bleeding into the central nervous system where the vasculature has been compromised a compound being used may or may not have the ability to cross the blood brain barrier. In certain embodiments, the compound being used to treat a head injury in a subject is able to cross the blood brain barrier. In other embodiments, the compounds is not able to cross the blood brain barrier. Certain desferrithiocin analogs with a 4′-polyether have been found in the CSF after systemic administration. See U.S. Provisional Application, filed Dec. 16, 2011, entitled “Uses of 4′-Desferrithiocin Analogs” by Bergeron.

Head injuries come in various forms and results from various causes. In certain embodiments, the injury is an injury to the head that penetrates the skull. In other embodiments, the head injury being treated is a closed head injury, which does penetrate the skull. Closed head injuries results from a variety of causes including accidents including vehicular accidents, falls, and assaults. Types of closed head injuries include concussions, brain contusions, diffuse axonal injury, and hematoma. In certain embodiments, the closed head injury being treated in the present invention include closed head injuries that result in blood outside the blood vessels of the brain.

The local accumulation of iron from the bleeding is thought to contribute to after effects of closed head injury. By assisting the clearance of iron from the brain the effects of the bleed are minimized.

In the treatment of closed head injury, the compound of Formula (I) or a pharmaceutical composition thereof may be administered systemically, for example, parenterally or orally. In certain embodiments, the compound or composition is administered orally. In other embodiments, the compound or composition is administered parenterally (e.g., intravenously).

The subject being treated for a head injury may be any type of animal. In certain embodiments, the animal is a mammal. In certain embodiments, the animal is a human. In certain embodiments, the animal is a domesticated animal (e.g., dog, cat, pig, cow). In certain embodiments, the animal is a research animal (e.g., mice, rat, dog, primate).

The exact amount of the compound of Formula (I) required to treat a head injury will vary from subject to subject, depending on the species, age, and general condition of the subject, the particular agent being administered, its mode of administration, and the like. The compound is preferably formulated in a dosage unit form for ease of administration and uniformity of dosage. It will be understood, however, that the total daily dose will be decided by a physician using sound medical judgment. The specific therapeutically effective dose level for any particular subject will depend upon a variety of factors including the severity of the head injury; the specific compound be administered; the age, body weight, general health, sex, and diet of the subject; the time of administration, route of administration, and rate of excretion of the particular compound being administered; the duration of the treatment; drugs used in combination or coincidental with the particular compound being administered; and like factors well known in the medical arts. In certain embodiments, the daily dosage of the compound of Formula (I) for the treatment of a head injury in a subject may range from 0.01 mg/kg to 200 mg/kg per unit dosage. In certain embodiments, the daily dosage ranges from 0.1 mg/kg to 100 mg/kg. In certain embodiments, the daily dosage ranges from 0.1 mg/kg to 50 mg/kg. In certain embodiments, the daily dosage ranges from 0.1 mg/kg to 20 mg/kg. In certain embodiments, the daily dosage ranges from 0.1 mg/kg to 10 mg/kg. In certain embodiments, the daily dosage ranges from 0.1 mg/kg to 1 mg/kg. In certain embodiments, the compound or a composition thereof may be administered once a day to multiple times per day. In certain embodiments, a fraction of the daily dose is administered once, twice, three times, or four times daily. In other embodiments, the compound of a composition thereof is administered every other day, every third day, every week, every other week, or every month. In certain embodiments, the inventive treatment is stopped once the head injury is resolved, or it is thought the inventive treatment would no longer be beneficial. In certain embodiments, the treatment is stopped once the bleeding has been resolved in a subject with a head injury.

### Treatment of Stroke

The present invention also provides for the treatment of stroke. The inventive treatment typically leads to a better and/or faster recovery from stroke. The stroke being treated may be either a ischemic stroke or a hemorrhagic stroke. In the treatment of an ischemic stroke, a compound of Formula (I) or composition thereof is administered to a subject to prevent or minimize the damage due to reperfusion injury after the blood supply to the affected part of the brain is restore. The compound is thought to prevent the generation of reactive oxygen species by either chelating iron responsible for the generation of such species and/or quenching such radical species when they do occur. In hemorrhagic stroke, the compound is thought to work by similar mechanisms although the sequestering of iron from the blood in the brain is probably the predominate mechanism by which the inventive treatment works. The mechanism of action of the compound of Formula (I) is similar to that in the treatment of head injury.

The compound being used in the treatment may have the ability to cross the blood brain barrier. In certain embodiments, the compound has the ability to cross the blood brain barrier. In other embodiments, the compound does not have the ability to cross the blood brain barrier. In certain embodiments, when the subject has been diagnosed with an ischemic stroke, the compound used in the treatment can pass through the blood brain barrier.

The present invention may be useful in treating a subject after the subject has been diagnosed with having a stroke, or a subject who is susceptible to having a stroke may be administered a compound of Formula (I) or composition thereof to prevent or minimize the stroke's effects. In certain embodiments, the compound is administered as quickly as possible after a subject has been diagnosed with having a stroke. In certain embodiments, the compound is administered to the subject while the stroke is still occurring. In certain embodiments, the compound or a composition thereof is administered to a subject who has a history of strokes or is susceptible to having a stroke because of the subject's underlying medical condition. The compound or composition thereof may be administered once or multiple times in the treatment of stroke.

In the treatment of stroke the compound of Formula (I) or a pharmaceutical composition thereof may be administered systemically, for example, parenterally or orally. In certain embodiments, the compound or composition is administered orally. In other embodiments, the compound or composition is administered parenterally (e.g., intravenously).

The subject being treated for stroke may be any type of animal. In certain embodiments, the animal is a mammal. In certain embodiments, the animal is a human. In certain embodiments, the animal is a domesticated animal (e.g., dog, cat, pig, cow). In certain embodiments, the animal is a research animal (e.g., mice, rat, dog, primate).

The exact amount of the compound of Formula (I) required to treat a stroke will vary from subject to subject, depending on the species, age, and general condition of the subject, the particular agent being administered, its mode of administration, and the like. The compound is preferably formulated in a dosage unit form for ease of administration and uniformity of dosage. It will be understood, however, that the total daily dose will be decided by a physician using sound medical judgment. The specific therapeutically effective dose level for any particular subject will depend upon a variety of factors including the severity of the stroke; the specific compound be administered; the age, body weight, general health, sex, and diet of the subject; the time of administration, route of administration, and rate of excretion of the particular compound being administered; the duration of the treatment; drugs used in combination or coincidental with the particular compound being administered; and like factors well known in the medical arts. In certain embodiments, the daily dosage of the compound of Formula (I) for the treatment of a stroke in a subject may range from 0.01 mg/kg to 200 mg/kg per unit dosage. In certain embodiments, the daily dosage ranges from 0.1 mg/kg to 100 mg/kg. In certain embodiments, the daily dosage ranges from 0.1 mg/kg to 50 mg/kg. In certain embodiments, the daily dosage ranges from 0.1 mg/kg to 20 mg/kg. In certain embodiments, the daily dosage ranges from 0.1 mg/kg to 10 mg/kg. In certain embodiments, the daily dosage ranges from 0.1 mg/kg to 1 mg/kg. In certain embodiments, the compound or a composition thereof may be administered once a day to multiple times per day. In certain embodiments, a fraction of the daily dose is administered once, twice, three times, or four times daily. In other embodiments, the compound or a composition thereof is administered every other day, every third day, every week, every other week, or every month. Typically the compound or composition thereof is not administered after it is no longer thought to be beneficial, for example, when all the bleeding has been cleared in a hemorrhagic stroke.

### Treatment of Inflammatory Bowel Disease

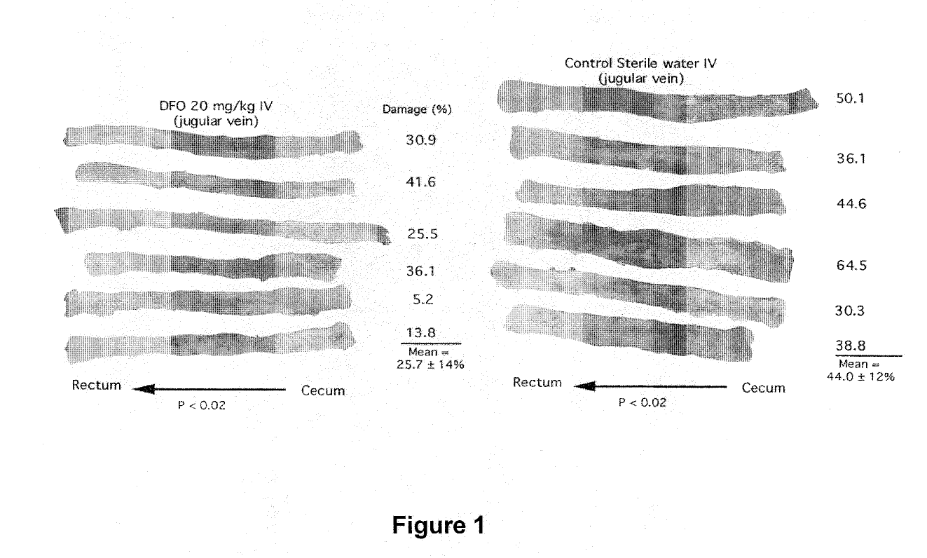

Reactive oxygen species have been implicated in the pathogenesis of inflammatory bowel disease (IBD). Grisham et al., “Neutophil-mediated mucosal injury. Role of reactive oxygen metabolites” Dig. Dis. Sci. 33:6S-15S, 1988; Allgayer “Clinical relevance of oxygen radicals in inflammatory bowel disease—facts and fashion” Klin. Wochenschr. 69:1001-1003, 1991; Ymamada et al. “Role of neutrophil-derived oxidants in the pathogenesis of intestinal inflammation” Klin. Wocheschr. 69:988-944, 1991; Babbs, “Oxygen radicals in ulcerative colitis” Free Radic. Biol. Med. 13:169-181, 1992. The present invention provides for the treatment of IBD. DFO, an iron chelator, has been discovered to prevent acetic acid-induced colitis in rats, an animal model of IBD. See FIG. 1 and Example 2. See, also, Bergeron et al., “Prevention of Acetic Acid-Induced Colitis by Desferrithiocin Analogs in a Rat Model” Digestive Diseases and Sciences, 48(2):399-407, February 2003. The compounds used in the inventive treatment are thought to prevent or eliminate the generation of reactive oxygen species or other longer-lived, more stable radicals that may be responsible for the tissue damage and inflammation seen in subjects with IBD. Another possible mechanism of action of the compounds useful in the invention is the chelation of metal, such as iron, which may contribute to the generation of reactive oxygen species, such as hydroxyl radicals and hydrogen peroxide, that cause cell damage.

The present invention may be useful in treating a subject diagnosed with IBD. The treatment may be used to treat the subject long term or may be used to treat a subject with a fare up of IBD. A therapeutically effective amount of a compound of Formula (I) or composition thereof is administered to a subject in need thereof to treat IBD. In certain embodiments, treatment with a compound of Formula (I) leads to reduced levels of reactive oxygen species in the intestines, specifically the intestinal mucosa. The compound or composition thereof may be administered to a subject once or multiple times in the treatment of IBD.

In the treatment of IBD, the compound of Formula (I) or a pharmaceutical composition thereof may be administered systemically, for example, parenterally or orally. In certain embodiments, the compound or composition is administered orally. In other embodiments, the compound or composition is administered parenterally (e.g., intravenously). In certain embodiments, the compound or a composition is administered rectally.

The subject being treated for IBD may be any type of animal. In certain embodiments, the animal is a mammal. In certain embodiments, the animal is a human. In certain embodiments, the animal is a domesticated animal (e.g., dog, cat, pig, cow). In certain embodiments, the animal is a research animal (e.g., mice, rat, dog, primate). In certain embodiments, the animal is used in animal model of IBD (e.g., acetic acid-induced colitis in rats; see Fedorak et al., “Misoprostol provides a colonic mucosal protective effect during acetic acid-induced colitis in rats” Gastroenterology 98:615-625, 1990; MacPherson et al., “Experimental production of diffuse colitis in rats” Digestion 17:135-150, 1978).

The exact amount of the compound of Formula (I) required to treat IBD will vary from subject to subject, depending on the species, age, and general condition of the subject, the particular agent being administered, its mode of administration, and the like. The compound is preferably formulated in a dosage unit form for ease of administration and uniformity of dosage. The total daily dose will be decided by a physician using sound medical judgment. The specific therapeutically effective dose level for any particular subject will depend upon a variety of factors including the severity of IBD; the specific compound be administered; the age, body weight, general health, sex, and diet of the subject; the time of administration, route of administration, and rate of excretion of the particular compound being administered; the duration of the treatment; drugs used in combination or coincidental with the particular compound being administered; and like factors well known in the medical arts. In certain embodiments, the daily dosage of the compound of Formula (I) for the prevention or treatment of reperfusion injury in a subject may range from 0.01 mg/kg to 200 mg/kg per unit dosage. In certain embodiments, the daily dosage ranges from 0.1 mg/kg to 100 mg/kg. In certain embodiments, the daily dosage ranges from 0.1 mg/kg to 50 mg/kg. In certain embodiments, the daily dosage ranges from 0.1 mg/kg to 20 mg/kg. In certain embodiments, the daily dosage ranges from 0.1 mg/kg to 10 mg/kg. In certain embodiments, the daily dosage ranges from 0.1 mg/kg to 1 mg/kg. In certain embodiments, the compound or a composition thereof may be administered once a day to multiple times per day. In certain embodiments, a fraction of the daily dose is administered once, twice, three times, or four times daily. In other embodiments, the compound or a composition thereof is administered every other day, every third day, every week, every other week, or every month.

### Treatment of Reperfusion Injury

The present invention also provides for the treatment of reperfusion injury. Reperfusion injury may occur in any area of the body where the blood supply has been compromised. In certain embodiments, the reperfusion injury being treated occurs in the heart. In other embodiments, the reperfusion injury occurs in the brain, for example, as discussed above in the context of a stroke. The inventive treatment minimizes reperfusion injury once the blood supply to the affects organ or tissue is restored. In the treatment or prevention of reperfusion injury, a compound of Formula (I) or composition thereof is administered to a subject who is suffering from ischemia of a tissue or organ. The compound of Formula (I) is thought to prevent the generation of reactive oxygen species by either chelating iron responsible for the generation of such species and/or quenching such radical species when they do occur.

The present invention may be useful in treating a subject after the subject has been diagnosed with ischemia of a particular organ or tissue. A therapeutically effective amount of a compound of Formula (I) or composition thereof is administered to a subject to prevent or minimize reperfusion injury. In certain embodiments, the compound is administered as quickly as possible after a subject has been diagnosed with ischemia. In certain embodiments, the compound is administered to the subject at risk of ischemia. In certain embodiments, the compound or a composition thereof is administered to a subject who is about to undergo a procedure that may lead to ischemia of an organ or tissue (e.g., cardiac surgery). In certain embodiments, the compound or a composition thereof is used to prevent reperfusion injury in a transplanted organ. In certain embodiments, the compound or composition thereof is used to perfuse an isolated organ being prepared for donation. The compound or composition thereof may be administered to a subject once or multiple times in the treatment of reperfusion injury.

In the prevention or treatment of reperfusion injury, the compound of Formula (I) or a pharmaceutical composition thereof may be administered systemically, for example, parenterally or orally. In certain embodiments, the compound or composition is administered orally. In other embodiments, the compound or composition is administered parenterally (e.g., intravenously). In certain embodiments, the compound or a composition is administered locally to the organ or tissue suffering from ischemia.

The subject being treated for reperfusion injury may be any type of animal. In certain embodiments, the animal is a mammal. In certain embodiments, the animal is a human. In certain embodiments, the animal is a domesticated animal (e.g., dog, cat, pig, cow). In certain embodiments, the animal is a research animal (e.g., mice, rat, dog, primate).

The exact amount of the compound of Formula (I) required to prevent or treat reperfusion injury will vary from subject to subject, depending on the species, age, and general condition of the subject, the particular agent being administered, its mode of administration, and the like. The compound is preferably formulated in a dosage unit form for ease of administration and uniformity of dosage. It will be understood, however, that the total daily dose will be decided by a physician using sound medical judgment. The specific therapeutically effective dose level for any particular subject will depend upon a variety of factors including the severity of the reperfusion injury; the specific compound be administered; the age, body weight, general health, sex, and diet of the subject; the time of administration, route of administration, and rate of excretion of the particular compound being administered; the duration of the treatment; drugs used in combination or coincidental with the particular compound being administered; and like factors well known in the medical arts. In certain embodiments, the daily dosage of the compound of Formula (I) for the prevention or treatment of reperfusion injury in a subject may range from 0.01 mg/kg to 200 mg/kg per unit dosage. In certain embodiments, the daily dosage ranges from 0.1 mg/kg to 100 mg/kg. In certain embodiments, the daily dosage ranges from 0.1 mg/kg to 50 mg/kg. In certain embodiments, the daily dosage ranges from 0.1 mg/kg to 20 mg/kg. In certain embodiments, the daily dosage ranges from 0.1 mg/kg to 10 mg/kg. In certain embodiments, the daily dosage ranges from 0.1 mg/kg to 1 mg/kg. In certain embodiments, the compound or a composition thereof may be administered once a day to multiple times per day. In other embodiments, the compound or a composition thereof is administered every other day, every third day, every week, every other week, or every month. Typically the compound or composition thereof is not administered after it is no longer thought to be beneficial, for example, when the risk of reperfusion injury is over.

### Pharmaceutical Compositions

The present invention also provides pharmaceutical compositions of Formula (I) for the treatment of macular degeneration, closed head injury, stroke, IBD, and reperfusion injury. After formulation with an appropriate pharmaceutically acceptable excipient in a desired dosage, the pharmaceutical compositions of this invention can be administered to humans and other animals orally, rectally, parenterally, intracisternally, intraperitoneally, topically, bucally, ocularly, or the like, depending on the disease or condition being treated. In certain embodiments, an agent of the invention may be administered orally or parenterally at dosage levels sufficient to deliver from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, a compound of Formula (I) is administered at a dose that is below the dose at which the agent causes non-specific effects. In certain embodiments, a compound of Formula (I) is administered at a dose that does not cause generalized immunosuppression in a subject.

Liquid dosage forms for oral and parenteral administration include, but are not limited to, pharmaceutically acceptable emulsions, microemulsions, solutions, suspensions, syrups, and elixirs. In addition to the active agents, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents. In certain embodiments for parenteral administration, agents of the invention are mixed with solubilizing agents such CREMOPHOR EL® (polyethoxylated castor oil), alcohols, oils, modified oils, glycols, polysorbates, cyclodextrins, polymers, and combinations thereof.

Injectable preparations, for example, sterile injectable aqueous or oleaginous suspensions, may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the compounds of this invention with suitable non-irritating excipients or carriers such as cocoa butter, polyethylene glycol, or a suppository wax which are solid at ambient temperature but liquid at body temperature and therefore melt in the rectum or vaginal cavity and release the active agent.

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used. Ophthalmic formulation, ear drops, and eye drops are also contemplated as being within the scope of this invention. Additionally, the present invention contemplates the use of transdermal patches, which have the added advantage of providing controlled delivery of an agent to the body. Such dosage forms can be made by dissolving or dispensing the agent in the proper medium. Absorption enhancers can also be used to increase the flux of the agent across the skin. The rate can be controlled by either providing a rate controlling membrane or by dispersing the agent in a polymer matrix or gel.

Additionally, the carrier for a topical formulation can be in the form of a hydroalcoholic system (e.g., liquids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems.

It will also be appreciated that the compounds of Formula (I) and pharmaceutical compositions thereof can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder, or they may achieve different effects (e.g., control of any adverse effects).

In still another aspect, the present invention also provides a pharmaceutical pack or kit comprising one or more containers filled with one or more of the ingredients of the pharmaceutical compositions of the present invention, and in certain embodiments, includes an additional approved therapeutic agent for use as a combination therapy. Optionally associated with such container(s) can be a notice in the form prescribed by a governmental agency regulating the manufacture, use, or sale of pharmaceutical products, which notice reflects approval by the agency of manufacture, use, or sale for human administration.

### EXAMPLES

In order that the invention described herein may be more fully understood, the following examples are set forth. It should be understood that these examples are for illustrative purposes only and are not to be construed as limiting this invention in any manner.

### Example 1

### Preparation of Sample Solutions

### Synthesis of the Desferrithiocin (DFT) Analogs

The desferrithiocin (DFT) analogs and salts thereof useful in the present invention can be prepared from readily available starting materials using methods known in the art. For example, (S)-3′-(HO)-DADFT-norPE (II-A) and (S)-3′-(HO)-DADFT-PE (III-A) may be synthesized according to the methods described in International PCT Application, PCT/US2008/003433, filed Mar. 14, 2008, published as WO 2008/115433, U.S. patent applications, U.S. Ser. No. 12/450,194, filed Dec. 14, 2009, published as US2010/0093812; and Bergeron et al., J. Med. Chem. (2006) 49:2772-2783, each of which are incorporated herein by reference.

### Preparation of Sample Solutions Containing Monosodium Salts of the DFT Analogs

The DFT analogs useful in the inventive methods were converted from the free acid form to the monosodium salt form. Water followed by one equivalent of sodium hydroxide was added to the DFT analog as a free acid. The resulting slurry was vortexed or sonicated until the DFT analog went into solution. More water was added, and the solution was vortexed or sonicated again. The formed yellow solution, having a pH about 7, was used as a sample solution in the following examples. It is preferred that a fresh sample solution of the DFT analog is made shortly before the solution is used in an assay.

### Example 2

### Prevention of Acetic Acid-Induced Colitis by Deferrithiocin Analogs in a Rat Model

Induction of Colitis.

Male Sprague-Dawley rats (250-350 g) were anesthetized with sodium pentobarbital, 55 mg/kg intraperitoneally. The abdomen was shaved and prepared for surgery. A midline incision was made, and the cecum and proximal colon were exteriorized. A reversible suture was placed at the junction of the cecum and proximal colon. The colon was rinsed with saline (10 ml), and the fluid and intestinal contents were gently expressed out the rectum. A gum-based rectal plug was inserted. The compound of interest, or distilled water in the control animals (2 ml), was injected intracolonically just distal to the ligature. The cecum and proximal colon were returned to the abdominal cavity; the compound was allowed to remain in the gut for 30 min. Then, the cecum and proximal colon were exteriorized again. The rectal plug was removed, and the drug was gently expressed out of the colon. Acetic acid (4%, 2 ml) was injected into the proximal colon over a 15- to 20-sec time period. The acid was allowed to remain in the gut until 1 min had passed (ie, 40-45 sec after the end of the acid administration). The no-acid control rats received distilled water (2 ml), which was administered in the same manner as was the acetic acid. Air (10 ml) was then injected into the proximal colon to expel the acid or water. The cecal/proximal colon ligature was removed, the gut was returned to the abdominal cavity, and the incisions were closed. The animals were allowed to recover overnight and were killed 24 hr later. The entire length of the colon was removed and assessed for damage both densitometrically and biochemically.

Quantitation of Acetic Acid-Induced Colitis.

Gross damage was quantitated using Photoshop-based image analysis (version 5.0, Adobe Systems, Mountain View, Calif., USA) on an Apple iMac computer. The Magic Wand tool in the Select menu of Photoshop was used to place the cursor on an area of obvious damage. The tolerance level of the Magic Wand tool was set at 30. The damaged areas were automatically selected by using the Similar command in the Select menu. Then, the Eyedropper tool was used to determine the range of the damage in the highlighted areas. Individual colon images were copied to a blank Photoshop page. The MagicWand tool, with a tolerance set to 100, was used to select all of the pixels in the colon sample. Then, the Histogram tool, which generates a graph in which each vertical line represents the number of pixels associated with a brightness level, was selected in the Image menu. The Red channel was then selected; the darker (damaged areas) appear on the left side of the histogram and the lighter (normal) areas are on the right side. The cursor was then placed on the histogram, the color range determined in an earlier step was selected, and the number of pixels encompassing that range and the percent damage were quantitated automatically.

Collection of Chelator Tissue Distribution Samples from Rodents.

Male Sprague-Dawley rats (250-350 g) were given the chelators orally at a dose of 300 pmol/kg. At times 0.5, 1, 2, 4 and 8 h after dosing (n=3) rats per time point, the animals were euthanized by exposure to CO₂ gas. Blood was obtained via cardiac puncture into vacutainers containing sodium citrate. The blood was centrifuged, and the plasma was separated for analysis. The liver, heart, pancreas, and kidneys were removed from the animals and frozen.

### Example 3

### Concentration of DFT Analogs in Rat Plasma and Cerebrospinal Fluid after Oral (PO) and Subcutaneous (SC) Doses

Adult male Sprague-Dawley rats (450-500 g) were used. The rats were not fasted. A sample solution of a monosodium salt of (S)-3′-(HO)-DADFT-norPE (II-A) or (S)-3′-(HO)-DADFT-PE (III-A) was administered to the rats at an oral or subcutaneous dose of 300 μmol/kg. Concentrations of the DFT analogs in the plasma and cerebrospinal fluid of the rats were measured at 0.5 hour, 1 hour, 2 hours, 4 hours, and 8 hours post administration. See Table 1 below.

TABLE 1
Concentration of DFT analogs in the plasma and cerebrospinal
fluid of rats treated with the DFT analogs

| | | | | | Concentration |
|---|---|---|---|---|---|
| | | | | Concentration | in |
| DFT | | | Time | in plasma | cerebrospinal |
| analog | Dose | LogPₐₚₚ | (h) | (μM) | fluid (μM) |
| II-A | 300 μmol/kg | −0.93 | 0.5 | 77 ± 10 | 0 ± 0 |
| | SC | | 1 | 66 ± 5 | 0 ± 0 |
| | | | 2 | 44 ± 2 | 0 ± 0 |
| | | | 4 | 7 ± 1 | 0 ± 0 |
| | | | 8 | trace | 0 ± 0 |
| III-A | 300 μmol/kg | −1.22 | 0.5 | 62 ± 24 | trace |
| | PO | | 1 | 46 ± 3 | 0 ± 0 |
| | | | 2 | 24 ± 5 | 0 ± 0 |
| | | | 4 | 13 ± 2 | 0 ± 0 |
| | | | 8 | trace | 0 ± 0 |

The foregoing written specification is considered to be sufficient to enable one skilled in the art to practice the present invention. The present invention is not to be limited in scope by the examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the claims. The advantages and objects of the present invention are not necessarily encompassed by each embodiment of the invention.

All publications, patent applications, and patents mentioned herein are hereby incorporated by reference in their entirety for disclosure of the teachings relevant to the present invention, as if each individual publication, patent application, or patent was specifically and individually indicated to be incorporated by reference. In case of the present specification and a document incorporated by reference including conflicting disclosure, the present specification shall control.

What is claimed is:

1. A method of treating macular degeneration in a subject comprising administering to the subject a therapeutically effective amount of a compound of Formula (I): 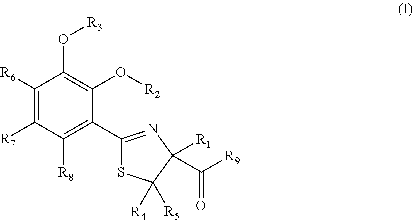
wherein:
R₁, R₄, and R₅ are each independently hydrogen, halogen, alkyl, or —OR₁₂;
R₂ is hydrogen, alkyl, or acyl;
R₃ is hydrogen, alkyl, or —[(CH₂)ₙ—O]ₓ—[(CH₂)ₙ—O]_{y}—R′;
R₆, R₇, and R₈ are each independently hydrogen, alkyl, arylalkyl, or —OR₁₀;
R₉ is —OR₁₁ or —SR₁₁;
R₁₀ is hydrogen, alkyl, or acyl;
R₁₁ is hydrogen or alkyl;
R₁₂ is hydrogen or alkyl;
R′ is alkyl;
each occurrence of n is independently an integer from 1 to 8, inclusive;
x is an integer from 1 to 8, inclusive; and
y is an integer from 0 to 8, inclusive;
or a pharmaceutically acceptable salt, tautomer, solvate, hydrate, or polymorph thereof.

2. The method of claim 1, wherein the compound is of the Formula (II):

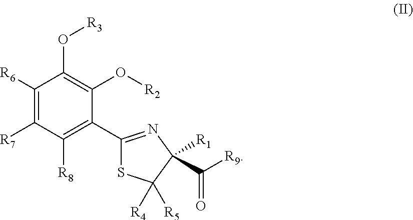

3. The method of claim 1, wherein R₁ is —CH₃.

4. The method of claim 1, wherein R₂ is hydrogen.

5. The method of claim 1, wherein R₃ is —CH₃.

6. The method of claim 1, wherein R₃ is —[(CH₂)ₙ—O]ₓ—R′; n is 2 to 4; and x is 1 to 4.

7. The method of claim 1, wherein R₃ is —[(CH₂)ₙ—O]ₓ—R′; n is 2; x is 3; and R′ is —CH₃.

8. The method of claim 1, wherein R₃ is —[(CH₂)ₙ—O]ₓ—R′; n is 2; x is 2; and R′ is —CH₃.

9. The method of claim 1, wherein R₄ and R₅ are each hydrogen.

10. The method of claim 1, wherein R₆, R₇, and R₈ are each hydrogen.

11. The method of claim 1, wherein R₂, R₄, R₅, R₆, R₇, and R₈ are each hydrogen.

12. The method of claim 1, wherein R₉ is —OH.

13. The method of claim 1, wherein the compound is of the Formula (II-A):

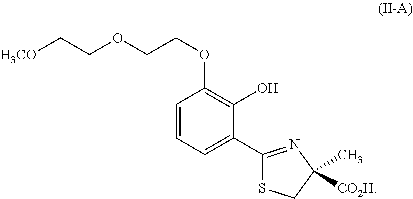 [SMILES: COCCOCCOc1cccc(C2=N[C@@](C)(C(=O)O)CS2)c1O]

14. The method of claim 1, wherein the compound is of the Formula (II-B) or (II-C):

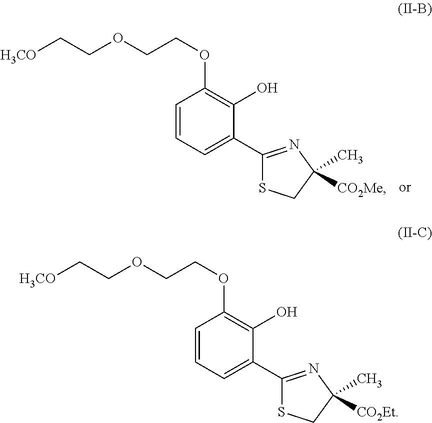 [SMILES: COCCOCCOc1cccc(C2=N[C@@](C)(C(=O)OC)CS2)c1O ; CCOC(=O)[C@@]1(C)CSC(c2cccc(OCCOCCOC)c2O)=N1]

15. (canceled)

16. The method of claim 1, wherein the compound is of the Formula (III-A):

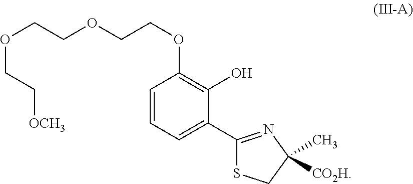 [SMILES: COCCOCCOCCOc1cccc(C2=N[C@@](C)(C(=O)O)CS2)c1O]

17. The method of claim 1, wherein the compound is of the Formula (III-B) or (III-C):

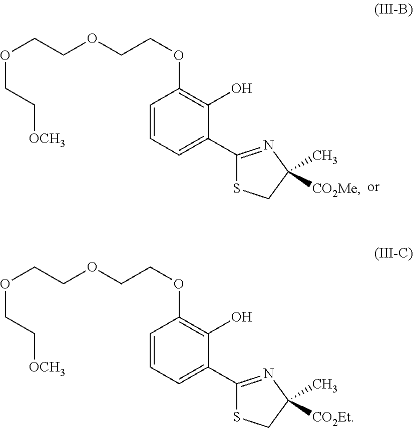 [SMILES: COCCOCCOCCOc1cccc(C2=N[C@@](C)(C(=O)OC)CS2)c1O ; CCOC(=O)[C@@]1(C)CSC(c2cccc(OCCOCCOCCOC)c2O)=N1]

18. (canceled)

19. The method of claim 1, wherein the compound is of the Formula (V-A), (V-B), or (V-C):

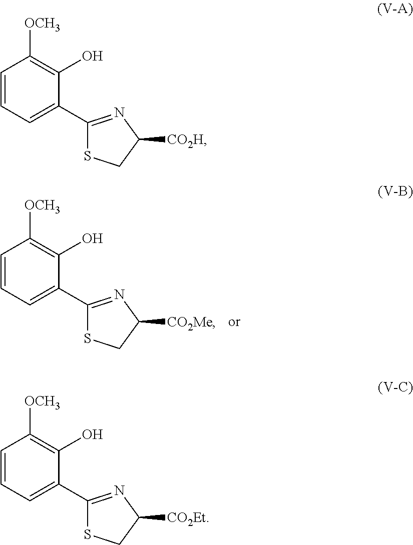 [SMILES: COc1cccc(C2=N[C@@H](C(=O)O)CS2)c1O ; COC(=O)[C@H]1CSC(c2cccc(OC)c2O)=N1 ; CCOC(=O)[C@H]1CSC(c2cccc(OC)c2O)=N1]

20-21. (canceled)

22. A method of treating a closed head injury in a subject comprising administering to the subject a therapeutically effective amount of a compound of Formula (I): 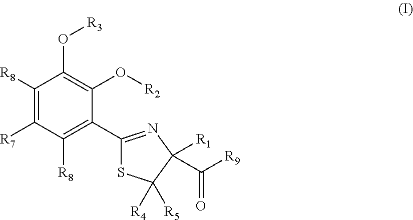
wherein:
R₁, R₄, and R₅ are each independently hydrogen, halogen, alkyl, or —OR₁₂;
R₂ is hydrogen, alkyl, or acyl;
R₃ is hydrogen, alkyl, or —[(CH₂)ₙ—O]ₓ—[(CH₂)ₙ—O]_{y}—R′;
R₆, R₇, and R₈ are each independently hydrogen, alkyl, arylalkyl, or —OR₁₀;
R₉ is —OR₁₁ or —SR₁₁;
R₁₀ is hydrogen, alkyl, or acyl;
R₁₁ is hydrogen or alkyl;
R₁₂ is hydrogen or alkyl;
R′ is alkyl;
each occurrence of n is independently an integer from 1 to 8, inclusive;
x is an integer from 1 to 8, inclusive; and
y is an integer from 0 to 8, inclusive;
or a pharmaceutically acceptable salt, tautomer, solvate, hydrate, or polymorph thereof.

23-46. (canceled)

47. A method of treating stroke in a subject comprising administering to the subject a therapeutically effective amount of a compound of Formula (I): 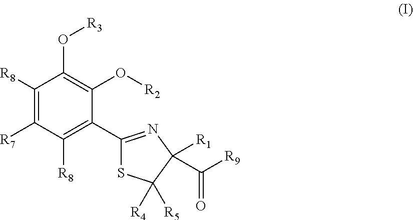
wherein:
R₁, R₄, and R₅ are each independently hydrogen, halogen, alkyl, or —OR₁₂;
R₂ is hydrogen, alkyl, or acyl;
R₃ is hydrogen, alkyl, or —[(CH₂)ₙ—O]ₓ—[(CH₂)ₙ—O]_{y}—R′;
R₆, R₇, and R₈ are each independently hydrogen, alkyl, arylalkyl, or —OR₁₀;
R₉ is —OR₁₁ or —SR₁₁;
R₁₀ is hydrogen, alkyl, or acyl;
R₁₁ is hydrogen or alkyl;
R₁₂ is hydrogen or alkyl;
R′ is alkyl;
each occurrence of n is independently an integer from 1 to 8, inclusive;
x is an integer from 1 to 8, inclusive; and
y is an integer from 0 to 8, inclusive;
or a pharmaceutically acceptable salt, tautomer, solvate, hydrate, or polymorph thereof.

48-69. (canceled)

70. A method of treating irritable bowel disease in a subject comprising administering to the subject a therapeutically effective amount of a compound of Formula (I): 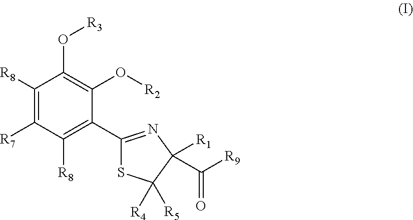
R₁, R₄, and R₅ are each independently hydrogen, halogen, alkyl, or —OR₁₂;
R₂ is hydrogen, alkyl, or acyl;
R₃ is hydrogen, alkyl, or —[(CH₂)ₙ—O]ₓ—[(CH₂)ₙ—O]_{y}—R′;
R₆, R₇, and R₈ are each independently hydrogen, alkyl, arylalkyl, or —OR₁₀;
R₉ is —OR₁₁ or —SR₁₁;
R₁₀ is hydrogen, alkyl, or acyl;
R₁₁ is hydrogen or alkyl;
R₁₂ is hydrogen or alkyl;
R′ is alkyl;
each occurrence of n is independently an integer from 1 to 8, inclusive;
x is an integer from 1 to 8, inclusive; and
y is an integer from 0 to 8, inclusive;
or a pharmaceutically acceptable salt, tautomer, solvate, hydrate, or polymorph thereof.

71-90. (canceled)

91. A method of treating reperfusion injury in a subject comprising administering to the subject a therapeutically effective amount of a compound of Formula (I): 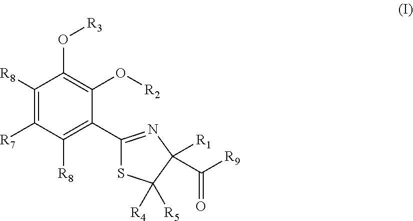
wherein:
R₁, R₄, and R₅ are each independently hydrogen, halogen, alkyl, or —OR₁₂;
R₂ is hydrogen, alkyl, or acyl;
R₃ is hydrogen, alkyl, or —[(CH₂)ₙ—O]ₓ—[(CH₂)ₙ—O]_{y}—R′;
R₆, R₇, and R₈ are each independently hydrogen, alkyl, arylalkyl, or —OR₁₀;
R₉ is —OR₁₁ or —SR₁₁;
R₁₀ is hydrogen, alkyl, or acyl;
R₁₁ is hydrogen or alkyl;
R₁₂ is hydrogen or alkyl;
R′ is alkyl;
each occurrence of n is independently an integer from 1 to 8, inclusive;
x is an integer from 1 to 8, inclusive; and
y is an integer from 0 to 8, inclusive;
or a pharmaceutically acceptable salt, tautomer, solvate, hydrate, or polymorph thereof.

92-116. (canceled)
